(19) United States
(12) Patent Application Publication
(10) Pub. No.: US 2014/0178274 A1
(43) Pub. Date: Jun. 26, 2014

(54) Method For Adjusting Temperature By Increasing Gas Molecular Density

(71) Applicant: Morningmoving Technology Co., Ltd., Hsinchu City (TW)

(72) Inventor: Chih-Horng Horng, Hsinchu City (TW)

(73) Assignee: Morningmoving Technology Co., Ltd., Hsinchu City (TW)

(21) Appl. No.: 13/969,472

(22) Filed: Aug. 16, 2013

(30) Foreign Application Priority Data
Dec. 22, 2012 (TW) 101149293

(51) Int. Cl. F28F 1/00; A61L 9/00

(52) U.S. Cl. CPC F28F 1/00; A61L 9/00
USPC 423/210; 165/108; 165/65

(57) ABSTRACT

The present invention relates to a method for adjusting temperature by increasing gas molecular density. The method includes: filling a chamber with at least one gas to a predetermined pressure, wherein the predetermined pressure is higher than 1 atm and lower than or equal to 50 atm; flowing the gas in the chamber to a temperature adjusting device, wherein the temperature adjusting device includes a cooler and a gas returning device; cooling the gas by the cooler; and returning the gas to the chamber by the gas returning device.

### CLAIM OF PRIORITY

This application claims the priority benefit of Taiwanese Application Serial Number 101149293, filed on Dec. 22, 2012. All disclosure of the Taiwanese application is incorporated herein by reference.

### BACKGROUND

1. Field

The present invention relates to a method for adjusting temperature, and more particularly to a method for adjusting temperature by increasing gas molecular density.

2. Related Art

A cooling process and a heating process usually play important roles in various industrial processes, and there is often a need for switching between such cooling and heating processes, e.g. switching between various processes requiring different temperatures. Over the switching period, it is needed to adjust the process temperature level to be adapted for the subsequent process; however, such adjustment is usually crucial to the process efficiency. For instance, in a situation where the temperature is from high to low, or from high to low and then back to high, etc., the faster the cooling and/or heating rate is, the higher the process efficiency is. In a conventional cooling process, an air cooling or water cooling process is frequently used, for example. However, since the cooling efficiency of such air cooling or water cooling process is so limited that a desirable result of rapid cooling can not be achieved, it is usually needed to expand an air cooling or water cooling equipment for improving the cooling efficiency, thus substantially increasing the equipment cost.

Therefore, for reducing the equipment cost, in a conventional air cooling process, for example, a non-circulating type temperature adjustment, in which a high temperature gas is exhausted directly and then a low temperature gas is fed to achieve the cooling result, is usually used. Although it appears that such non-circulating type temperature adjustment can reduce the equipment cost, this approach would in fact raise the total manufacturing cost since the original high temperature gas has to be exhausted out and an additional low temperature gas must be fed from the outside, thus increasing the gas cost for the inability to use the original gas effectively.

Under the forementioned circumstance, if it is further desired to have the process temperature back to (or even higher than) the level before it drops, the low temperature gas has to be exhausted out and an additional high temperature gas must be fed from the outside. In addition, in order to have the process temperature rapidly back to (or even higher than) the level before it drops, it is necessary to expand the heating equipments. Therefore, the conventional heating process will encounter the same problem as the cooling process does.

### SUMMARY OF THE INVENTION

The present invention improves the cooling and heating efficiencies by increasing gas molecular density (the pressure produced thereby being lager than 1 atm), i.e. increasing the number of the gas molecules to improve the temperature adjusting efficiency for a result of rapid cooling and heating. Therefore, the present invention may substantially decrease the equipment cost in comparison with the conventional cooling and heating processes. Further, the present invention utilizes a circulating manner (that is, the gas may be used repeatedly) to save the total consumption of the gas such that a goal to decrease the manufacturing cost is further reached.

According to one embodiment of the present invention, a method for adjusting temperature is provided, the method including: filling a chamber with a predetermined number of gas molecules, which keep the chamber in a predetermined pressure, by at least one gas, wherein the predetermined pressure is larger than 1 atm and less than or equal to 50 atm; flowing the gas in the chamber to a temperature adjusting device, wherein the temperature adjusting device includes a cooler and a gas returning device; cooling the gas by the cooler; and returning the gas back to the chamber by the gas returning device.

According to another embodiment of the present invention, a method for adjusting temperature is provided, the method including: filling a chamber with a predetermined number of gas molecules, which keep the chamber in a predetermined pressure, by at least one gas, wherein the predetermined pressure is larger than 1 atm and less than or equal to 50 atm; flowing the gas in the chamber to a temperature adjusting device, wherein the temperature adjusting device includes a cooler and a relief element; cooling the gas by the cooler; and returning the gas back to the chamber by a pressure difference created between the temperature adjusting device and the chamber through enabling the relief element and keeping filling the chamber with the gas.

Other aspects and advantages of the present invention will be apparent from the following detailed description in conjunction with the accompanying drawings by way of exemplifying the principle of the present invention. Further, a well-known device or principle will not be described herein to avoid any unnecessary obscuration to the present invention.

### BRIEF DESCRIPTION OF THE DRAWINGS

In the accompanying figures of the present invention, the same element is denoted with the same reference numeral.

FIG. 1 shows a schematic illustration of a processing system for adjusting temperature in accordance with one embodiment of the present invention.

FIG. 2 shows a schematic illustration of a processing system for adjusting temperature in accordance with another embodiment of the present invention.

FIG. 3 shows a schematic illustration of a processing system for adjusting temperature in accordance with another embodiment of the present invention.

FIG. 4 shows a schematic illustration of a processing system for adjusting temperature in accordance with another embodiment of the present invention.

FIG. 5 shows a schematic illustration of a processing system for adjusting temperature in accordance with another embodiment of the present invention.

FIG. 6 shows a schematic illustration of a processing system for adjusting temperature in accordance with another embodiment of the present invention.

### DETAILED DESCRIPTION OF PREFERRED EMBODIMENTS

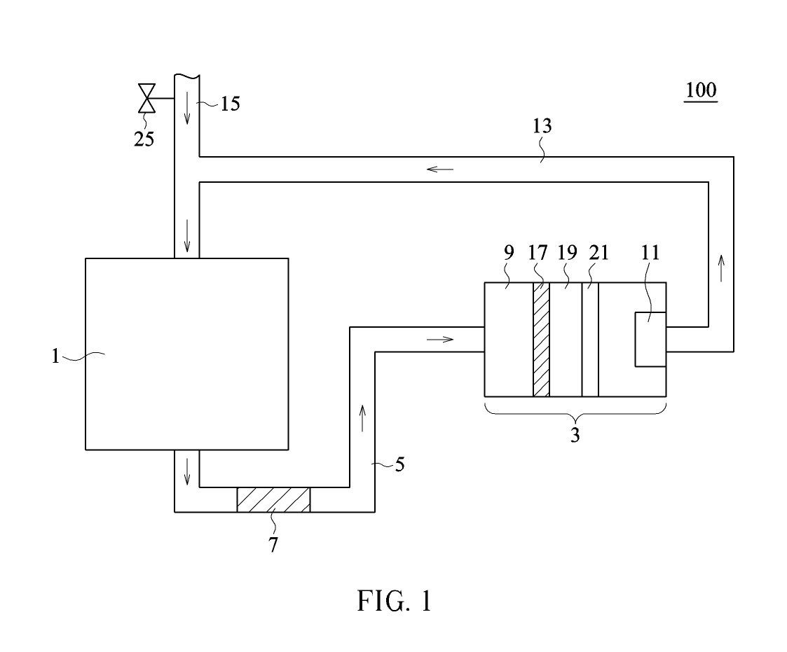

According to one embodiment of the present invention, FIG. 1 shows a schematic illustration of a processing system for adjusting temperature 100. The processing system for adjusting temperature 100 may include a processing chamber 1 and a temperature adjusting device 3. The processing chamber 1 is connected to the temperature adjusting device 3 through a gas pipeline 5. A gas in the processing chamber 1 may flow into the temperature adjusting device 3 through the gas pipeline 5. One or more filters 7 may be further provided between the processing chamber 1 and the temperature adjusting device 3 to filter the gas flowing out of the processing chamber 1. The temperature adjusting device 3 may include a cooler 9 and a gas returning device 11. The cooler 9 may be used to cool the gas from the chamber 1, and the gas returning device 11 may be used to return the gas back to the chamber 1. In one embodiment of the present invention, the gas returning device 11 is a fan, and in another embodiment of the present invention, the gas returning device 11 is a pump. The gas returning device 11 may create an external force (pressure difference) to force the gas to return to the processing chamber 1. The processing chamber 1 may be a processing chamber which can endure high pressure and high temperature.

The temperature adjusting device 3 may be connected to a gas feeding pipeline 15 through a gas pipeline 13. The temperature adjusting device 3 may return the gas back to the processing chamber 1 through the gas feeding pipeline 15 connected to the gas pipeline 13. In one embodiment of the present invention, the gas pipeline 13 may be not connected to the gas feeding pipeline 15, and the gas returns to the processing chamber 1 directly through the gas pipeline 13.

The gas feeding pipeline 15 may be connected to one or more gas sources (not shown). For example, such gas source may be a filling gas source for providing at least one filling gas, or a filling and heating gas source for providing at least one filling and heating gas. When the filling and heating gas is provided, the processing chamber 1 is filled with gas and heated. In one embodiment of the present invention, the processing chamber 1 may include a heater (not shown) to heat the gas inside such that the processing chamber 1 is heated. A valve 25 may be provided in the gas feeding pipeline 15 to control an input of gas from the gas source. Through the gas feeding pipeline 15, at least one gas from the gas source is fed into the chamber 1 such that the chamber 1 is filled with a predetermined number of gas molecules, which keep the chamber 1 at a predetermined pressure. In one embodiment of the present invention, the predetermined pressure may be larger than 1 atm and less than or equal to 50 atm.

The temperature adjusting device 3 may further include one or more filters 17 for filtering the gas from the processing chamber 1. Further, the temperature adjusting device 3 may further include a heater 19 for heating the gas after such gas from the processing chamber 1 was cooled by the cooler 9. In general, the gas leaving the chamber 1 may carry gaseous process chemicals. While the gas is cooled by the cooler 9, the gaseous process chemicals carried by the gas may condense and remain on the filter 17 and thus be filtered out. In one embodiment of the present invention, the temperature adjusting device 3 may further include a catalytic converter 21 to process the gas when heated, such as purifying the gas by the catalytic converter 21 through a catalytic reaction (e.g. converting the components of CO, HC, NOₓ, etc. in the gas into a harmless gas to human body as CO₂, H₂O, N₂, 0₂, etc.) The catalytic converter 21 may, for example, include rhodium, platinum, palladium, and so on as a catalyst. The catalytic converter 21 may be a two-way or three-way catalytic converter, or a combination of a two-way and a three-way catalytic converter.

According to the embodiment in FIG. 1, a method for adjusting temperature by increasing gas molecular density of the present invention may include the following steps: filling the chamber 1 with a predetermined number of gas molecules, which keep the chamber 1 at a predetermined pressure, by introducing at least one gas, wherein the predetermined pressure is larger than 1 atm and less than or equal to 50 atm; flowing the gas in the chamber 1 to the temperature adjusting device 3, wherein the temperature adjusting device 3 includes the cooler 9 and the gas returning device 11; cooling the gas by the cooler 9; and returning the gas back to the chamber 1 by the gas returning device 11.

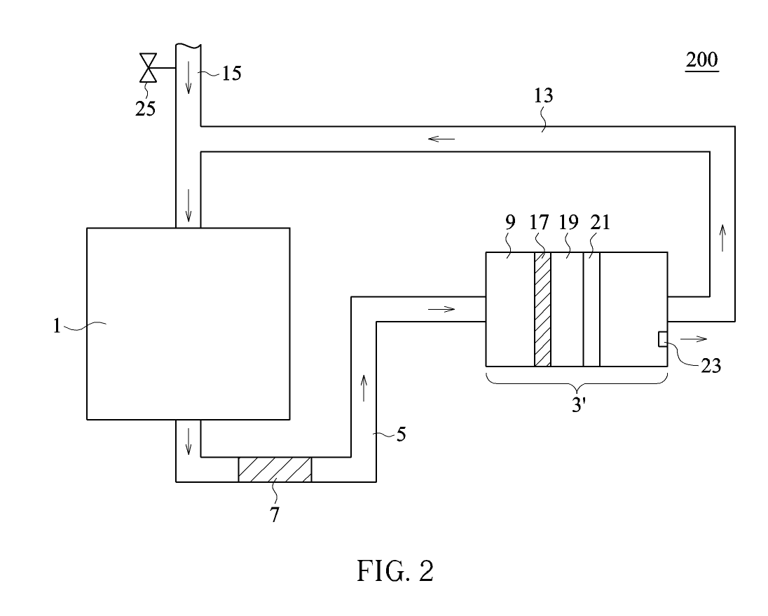

Another embodiment of the present invention is illustrated below with reference to FIG. 2, which shows a schematic illustration of a processing system for adjusting temperature 200.

The processing system for adjusting temperature 200 in FIG. 2 is similar to the processing system for adjusting temperature 100 in FIG. 1, except that a temperature adjusting device 3′ of the processing system for adjusting temperature 200 in FIG. 2 includes a pressure-relief element 23.

According to the embodiment in FIG. 2, a method for adjusting temperature by increasing gas molecular density of the present invention may include: filling the chamber 1 with a predetermined number of gas molecules, which keep the chamber 1 at a predetermined pressure, by introducing at least one gas, wherein the predetermined pressure is larger than 1 atm and less than or equal to 50 atm; flowing the gas in the chamber 1 to the temperature adjusting device 3′, wherein the temperature adjusting device 3′ includes the cooler 9 and the pressure-relief element 23; cooling the gas by the cooler 9; and returning the gas back to the chamber 1 by a pressure difference created between the temperature adjusting device 3′ and the chamber 1 through opening the pressure-relief element 23 and keeping filling the chamber 1 with gas. The flow rate of the gas released from the pressure-relief element 23 is less than the flow rate of the gas entering the temperature adjusting device 3′. Most of the gas entering the temperature adjusting device 3′ may return to the chamber 1 through the gas feeding pipeline 15 connected to the gas pipeline 13. In one embodiment of the present invention, the gas pipeline 13 may not be connected to the gas feeding pipeline 15, and the gas may return to the chamber 1 directly through the gas pipeline 13. In terms of the pressure difference, there is not particular limit to the range of its numerical value, as long as it can make the gas return to the processing chamber 1. In one embodiment of the present invention, a relief valve (not shown) may be used as the pressure-relief element 23. In other embodiments of the present invention, the pressure-relief element 23 may be any gate or opening having opening and closing functions. In the embodiments shown in FIGS. 1 and 2, one or more filters (not shown) may be provided on a path from the gas pipeline 13 to the gas feeding pipeline 15 to filter the gas leaving the temperature adjusting device 3 (3′).

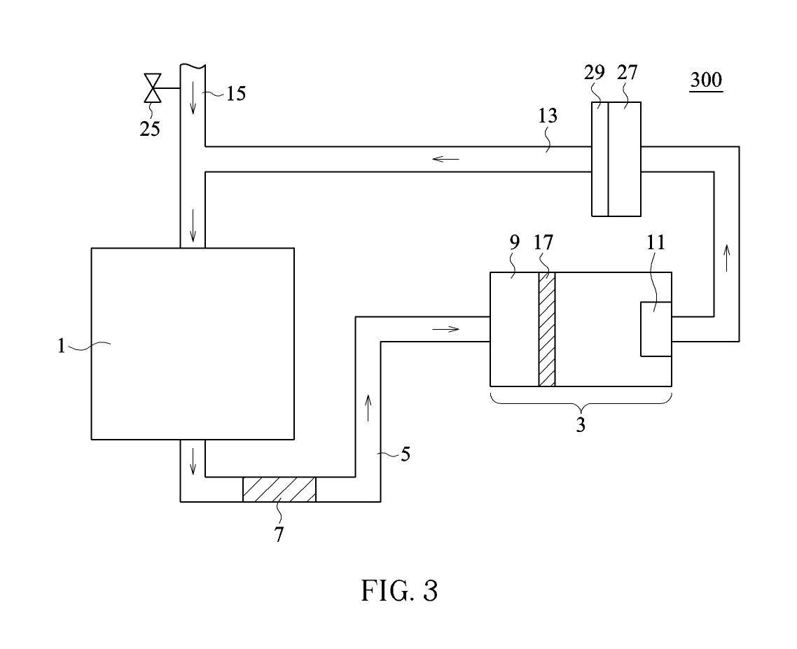

FIG. 3 shows a schematic illustration of a processing system for adjusting temperature 300 in accordance with another embodiment of the present invention. The processing system for adjusting temperature 300 in FIG. 3 is similar to the processing system for adjusting temperature 100 in FIG. 1, except that the temperature adjusting device 3 of the processing system for adjusting temperature 300 in FIG. 3 does not include the heater 19 and the catalytic converter 21, and a heater 27 and a catalytic converter 29 are independently provided outside the temperature adjusting device 3 with an external connection thereto so as to make replacement or maintenance of the heater 27 and the catalytic converter 29 more convenient. The heater 27 and the catalytic converter 29 may be positioned on a path of the gas pipeline 13.

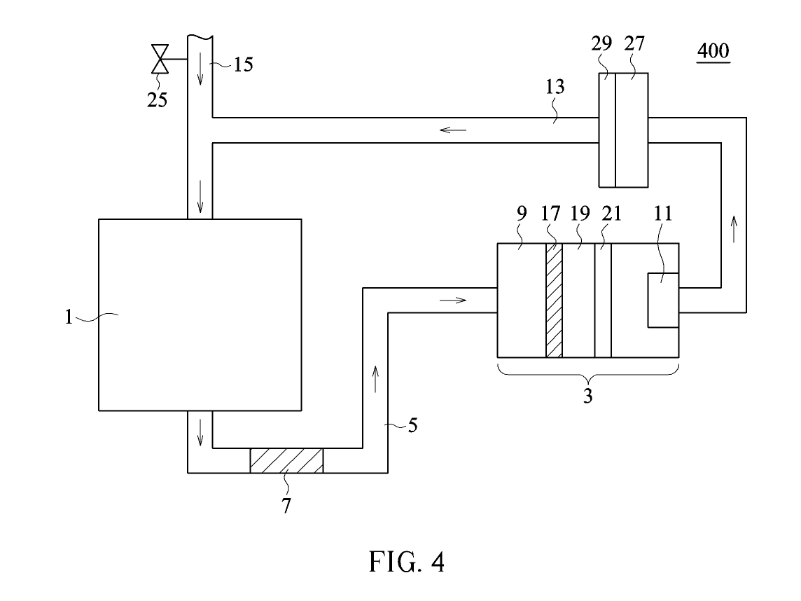

FIG. 4 shows a schematic illustration of a processing system for adjusting temperature 400 in accordance with another embodiment of the present invention. The processing system for adjusting temperature 400 in FIG. 4 is similar to the processing system for adjusting temperature 100 in FIG. 1, except that in the processing system for adjusting temperature 400 in FIG. 4, a heater 27 and a catalytic converter 29 are additionally provided outside the temperature adjusting device 3 with an external connection thereto so as to further heat and/or process the gas leaving the temperature adjusting device 3.

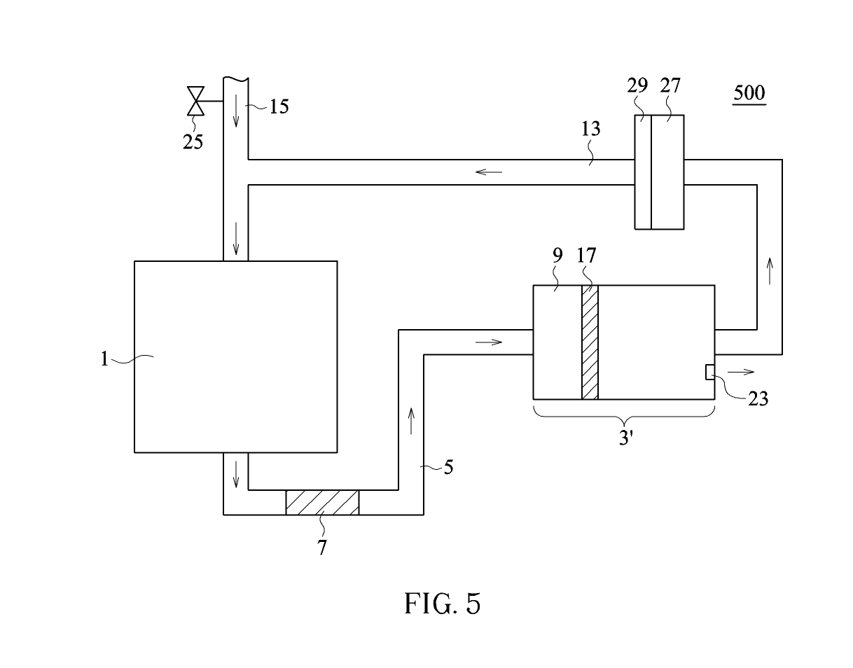

FIG. 5 shows a schematic illustration of a processing system for adjusting temperature 500 in accordance with another embodiment of the present invention. The processing system for adjusting temperature 500 in FIG. 5 is similar to the processing system for adjusting temperature 200 in FIG. 2, except that the temperature adjusting device 3′ of the processing system for adjusting temperature 500 in FIG. 5 does not include the heater 19 and the catalytic converter 21, and a heater 27 and a catalytic converter 29 are independently provided outside the temperature adjusting device 3′ with an external connection thereto so as to make replacement or maintenance of the heater 27 and the catalytic converter 29 more convenient. The heater 27 and the catalytic converter 29 may be positioned on a path of the gas pipeline 13.

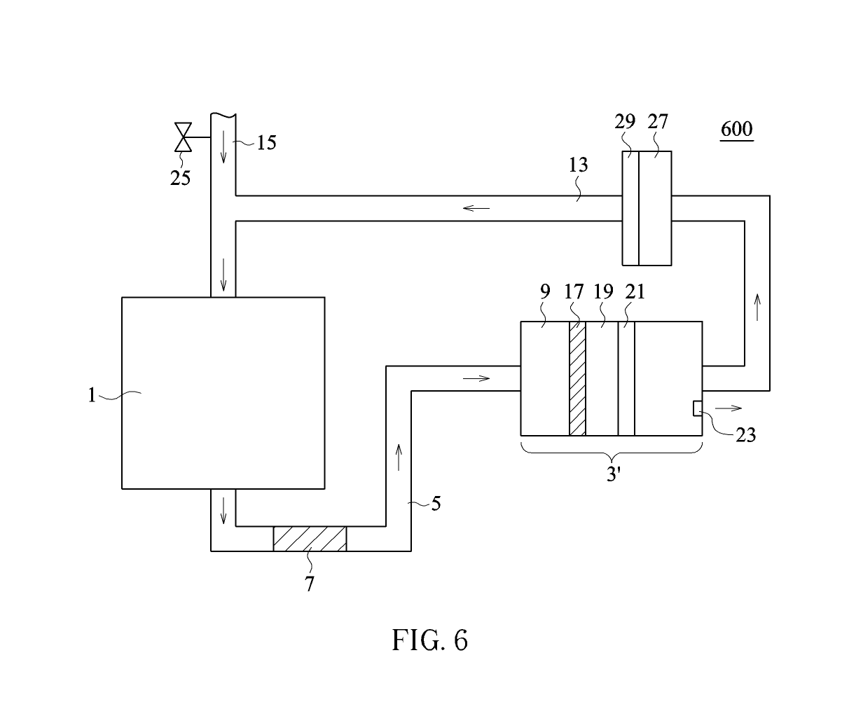

FIG. 6 shows a schematic illustration of a processing system for adjusting temperature 600 in accordance with another embodiment of the present invention. The processing system for adjusting temperature 600 in FIG. 6 is similar to the processing system for adjusting temperature 200 in FIG. 2, except that in the processing system for adjusting temperature 600 in FIG. 6, a heater 27 and a catalytic converter 29 are additionally provided outside the temperature adjusting device 3′ with an external connection thereto so as to further heat and/or process the gas leaving the temperature adjusting device 3′.

In the embodiments shown in FIGS. 1 to 6, one may use the heater 19 and/or the heater 27 to make the temperature of the gas returning to the chamber 1 reaches a temperature equal to or higher than the temperature inside the processing chamber 1.

In the embodiments shown in FIGS. 3 to 6, after leaving the temperature adjusting device 3 (3′) and before returning to the chamber 1, the gas may be heated by the heater 27. Further, for filtering the gas leaving the temperature adjusting device 3 (3′), one or more filters (not shown) may be provided on a path from the gas pipeline 13 to the gas feeding pipeline 15 and/or on a path from the temperature adjusting device 3 (3′) to the heater 27 and the catalytic converter 29.

Furthermore, in the embodiments shown in FIGS. 3 and 5, the heater 27 may be provided inside the temperature adjusting device 3 (3′) and the catalytic converter 29 may be provided outside the temperature adjusting device 3 (3′). Therefore, after leaving the temperature adjusting device 3 (3′) and before returning to the chamber 1, the gas heated by the heater 27 which is provided inside the temperature adjusting device 3 (3′) may be processed by the catalytic converter 29 which is provided outside the temperature adjusting device 3 (3′).

The present invention improves the cooling and heating efficiencies by increasing gas molecular density (the pressure produced thereby being lager than 1 atm), i.e. increasing the number of the gas molecules to improve the temperature adjusting efficiency for a result of rapid cooling and heating. Therefore, the present invention may substantially decrease the equipment cost in comparison with the conventional air cooling or water cooling process. Further, the present invention utilizes a circulating manner (that is, the gas may be used repeatedly) to save the total consumption of the gas such that a goal to decrease the manufacturing cost is further reached.

While the present invention has been described in detail with reference to the preferred embodiments and the drawings, one skilled in the art may implement various modifications, alterations, and equivalents without departing from the true spirit and scope of the present invention, and such modifications, alterations, and equivalents will fall in the scope of the appended claims.

What is claimed is:

1. A method for adjusting temperature by increasing gas molecular density, including:
filling a chamber with at least one gas to a predetermined pressure, wherein the predetermined pressure is larger than 1 atm and less than or equal to 50 atm;
flowing the gas in the chamber to a temperature adjusting device, wherein the temperature adjusting device includes a cooler and a gas returning device;
cooling the gas by the cooler; and
returning the gas back to the chamber by the gas returning device.

2. A method for adjusting temperature by increasing gas molecular density, including:
filling a chamber with at least one gas to a predetermined pressure, wherein the predetermined pressure is larger than 1 atm and less than or equal to 50 atm;
flowing the gas in the chamber to a temperature adjusting device, wherein the temperature adjusting device includes a cooler and a pressure-relief element;
cooling the gas by the cooler; and
returning the gas back to the chamber by a pressure difference created between the temperature adjusting device and the chamber through enabling the pressure-relief element and keeping filling the chamber with the gas.

3. The method for adjusting temperature according to claim 1, wherein the gas returning device is a fan.

4. The method for adjusting temperature according to claim 1, wherein the gas returning device is a pump.

5. The method for adjusting temperature according to claim 1, wherein the temperature adjusting device further includes a heater to heat the gas after it was cooled.

6. The method for adjusting temperature according to claim 2, wherein the temperature adjusting device further includes a heater to heat the gas after it was cooled.

7. The method for adjusting temperature according to claim 5, wherein the temperature adjusting device further includes a catalytic converter to process the gas when heated.

8. The method for adjusting temperature according to claim 6, wherein the temperature adjusting device further includes a catalytic converter to process the gas when heated.

9. The method for adjusting temperature according to claim 5, further including:
processing the gas by a catalytic converter after the gas leaves the temperature adjusting device and before it returns to the chamber.

10. The method for adjusting temperature according to claim 6, further including:
processing the gas by a catalytic converter after the gas leaves the temperature adjusting device and before it returns to the chamber.

11. The method for adjusting temperature according to claim 1, further including:
heating the gas by a heater after the gas leaves the temperature adjusting device and before it returns to the chamber.

12. The method for adjusting temperature according to claim 2, further including:
heating the gas by a heater after the gas leaves the temperature adjusting device and before it returns to the chamber.

13. The method for adjusting temperature according to claim 11, wherein after the gas leaves the temperature adjusting device and before it returns to the chamber, a catalytic converter is used to process the gas when heated.

14. The method for adjusting temperature according to claim 12, wherein after the gas leaves the temperature adjusting device and before it returns to the chamber, a catalytic converter is used to process the gas when heated.
